(19) United States
(12) Patent Application Publication
(10) Pub. No.: US 2023/0363815 A1
(43) Pub. Date: Nov. 16, 2023

(54) ELECTRODE ASSEMBLY

(71) Applicant: GYRUS MEDICAL LIMITED, Cardiff (GB)

(72) Inventor: James DICKSON, Cardiff (GB)

(73) Assignee: GYRUS MEDICAL LIMITED, Cardiff (GB)

(21) Appl. No.: 18/143,674

(22) Filed: May 5, 2023

Related U.S. Application Data
(60) Provisional application No. 63/340,108, filed on May 10, 2022.

(51) Int. Cl. A61B 18/14

(52) U.S. Cl. CPC A61B 18/14; A61B 2018/00589

(57) ABSTRACT

The present disclosure relates to an electrode assembly provided with a lead-in feature that gradually increases the diameter of the outer shaft to that of the insulation sleeve. In doing so, there is no blunt edge formed by the insulation used to secure the electrode assembly to the instrument handle. This prevents a user catching this edge on the cannula, therefore reducing the force needed to insert the device, and the risk of causing damage to the insulation or entry site of the patient.

### TECHNICAL FIELD

The present invention relates to electrosurgical instruments. More specifically, the present invention relates to an end effector for an electrosurgical instrument, the end effector comprising an electrode assembly.

### BACKGROUND TO THE INVENTION AND PRIOR ART

Surgical instruments, including radio frequency (RF) electrosurgical instruments, have become widely used in surgical procedures where access to the surgical site is restricted to a narrow passage, for example, in minimally invasive “keyhole” surgeries.

RF electrosurgical instruments typically include an active electrode which forms a distal RF tip of the instrument, and a return electrode. The distal RF tip provides tissue ablation and/or coagulation effects at a surgical site when a RF power signal is delivered to the electrodes.

In prior art RF bi-polar electrode assemblies, the return electrode is often configured as an outer steel shaft that is mechanically coupled to the handle of the instrument. In order to limit the exposed return electrode to just the distal end of instrument assembly, an insulating material is usually provided over the return electrode. This is typically a heat-shrink style material such as polyvinylidene fluoride (PVDF), or a rigid lumen configured to tightly slide over the outer shaft.

FIGS. 2A-B illustrate an example of a distal RF tip 20 in a prior art device, comprising an active electrode 22 retained within an insulator 24 and a return electrode 26 in the form of part of an outer shaft. An insulating sleeve 28 is provided around the return electrode 28 to limit the exposed area of the return electrode 26 to only the distal end of the instrument assembly, that is, so that the return electrode 26 does not need to extend all the way down to the hand piece of the instrument. This arrangement results in a blunt edge 30 at the point where the insulating sleeve 28 begins, such that there is an abrupt change to the outside diameter of the outer shaft. This blunt edge 30 has a number of disadvantages. Firstly, it can catch on a cannula or percutaneous port entry site, which can impede progress of the instrument into the body of a patient, and could increase port-site trauma. Secondly, the interaction of this blunt edge 30 with a rigid cannula can cause damage to the softer instrument insulation 28. In some cases, this could lead to material being scraped away from the instrument and being left in the patient.

As such, there is a need to further improve the configuration of the return electrode and its definition at the distal end of the device.

### SUMMARY OF THE INVENTION

In known RF electrosurgical instruments that implement a return electrode in the form of an outer shaft, the insulating sleeve used to define the end region of the return electrode typically leaves a blunt edge that can catch on other surfaces during use. This can result in damage to the entry site on the patient, and if the material erodes a sufficient amount, can lead to material being left in the patient after the instrument is removed, or an increase in the RF current density if pinholes are created in the insulating sleeve. The present disclosure therefore seeks to address this problem by providing a lead-in feature on the electrode assembly, to gradually increase the diameter of the outer shaft to that of the insulation sleeve. This would prevent a user catching this edge on the cannula, and therefore reduce the force needed to insert the device, and reduce the risk of damaging the insulation or hurting the patient.

A first aspect of the present disclosure provides an end effector for an electrosurgical instrument, comprising an electrode assembly for delivering a radio-frequency (RF) power signal to a surgical site, the electrode assembly comprising an active electrode and a return electrode, wherein the electrode assembly comprises at least a first portion having a first outer diameter and a second portion having a second outer diameter, the first outer diameter being larger than the second outer diameter; and an insulating sleeve configured to be received by the second portion of the electrode assembly, the insulating sleeve having an outer diameter substantially the same as the first outer diameter.

For example, the return electrode may comprise the first portion having the first outer diameter and the second portion having the second diameter. As such, when the insulating sleeve is placed around the second portion of the return electrode, the outer surface of the insulating sleeve lies substantially level with the outer surface of the first portion of the return electrode, thereby forming a smooth outer surface at the junction between the first portion and the insulating sleeve. As such, there is no lip or edge formed by the end of the insulating sleeve on which things may catch, thus reducing the force needed to insert the device.

The return electrode may comprise a lead-in feature between the first portion and the second portion to thereby provide a difference in outer diameter. That is to say, the return electrode comprises a feature that creates the difference in diameter between the first portion and the second potion.

For example, the return electrode may comprise a protruding edge formed between the first portion and the second portion. The protruding edge may extend around at least a portion of a circumference of the return electrode. For example, the protruding edge may extend around up to about 75% of the circumference of the return electrode. The remaining circumference of the return electrode may then provide a surface for receiving the active electrode or a ceramic insulator.

An end of the insulating sleeve may be configured to mate with a surface of the protruding edge. That is to say, edge may comprise a surface pointing in the proximal direction (i.e., away from the active electrode and towards the handle of the instrument); when the insulating sleeve is formed around the second portion of the return electrode, the end of that insulating sleeve abuts the surface of the protruding edge.

In some arrangements, the first portion of the return electrode may comprise a sloped surface, the sloped surface forming the protruding edge. That is to say, the outer diameter of the first portion gradually increases, such that the outer diameter in the region of the protruding edge substantially matches the outer diameter of the insulating sleeve.

It will of course be appreciated that the first and second portions may be provided by different parts of the electrode assembly. For example, the return electrode may provide the second portion, whilst the first portion with the larger diameter may be provided by a separate lead in component arranged on the electrode assembly, for example, on a ceramic insulating component. That is, the electrode assembly may further comprise an insulating element arranged between the active electrode and the return electrode, wherein the insulating element comprises the first portion having the first outer diameter and the return electrode comprises the second portion having the second outer diameter.

The insulating element may be formed of a ceramic or a polymer.

The end effector may also comprise a rotary shaver arrangement. In such cases, the rotary shaving arrangement may be operably connected to drive componentry to drive the rotary shaver to operate in use.

The insulating sleeve may be heat-shrink wrapped around the second portion of the electrode assembly. The insulating sleeve may comprise a polymer material. For example, the polymer material may comprise polyvinylidene fluoride.

The return electrode may be manufactured by a process of metal injection moulding. This allows the lead-in feature (e.g., the protruding edge) to be easily moulded into the return electrode component without adding significant cost to the return electrode or overall assembly of the end effector.

The active electrode may comprise an aperture for providing access to a suction channel extending through the insulating element to a lumen configured to carry fluid from the surgical site. The lumen may be connectable to a suction tube for connecting to a suction source.

The active electrode and/or return electrode may be formed from a metal. For example, the metal may be any one of copper, stainless steel, tungsten or an alloy of tungsten and platinum.

The active electrode and the return electrode may be connectable to a RF power source.

A further aspect provides an electrosurgical instrument, comprising a hand-piece, one or more user-operable buttons on the handpiece for operably controlling the instrument, and an operative shaft, having RF electrical connections, and drive componentry for an end effector, the electrosurgical instrument further comprising an end effector as described above, the active electrode and the return electrode being connected to the RF electrical connections.

Another aspect provides an electrosurgical system, comprising an RF electrosurgical generator, a suction source, and an electrosurgical instrument as described above, the arrangement being such that in use the RF electrosurgical generator supplies an RF coagulation or ablation signal via the RF electrical connections to the active electrode and the return electrode.

Yet a further aspect of the present invention may provide a method for processing an instrument for surgery, the method comprising obtaining an electrosurgical instrument and/or end effector described herein, sterilizing the electrosurgical instrument and/or end effector, and storing the electrosurgical instrument and/or end effector in a sterile container.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 shows an example of the electrosurgical instrument system comprising an RF electrosurgical instrument according to the present invention;

FIGS. 2A-2B illustrate part of an end effector known in the prior art;

FIG. 3 illustrates an example of an end effector in accordance with the present invention;

FIG. 4 further illustrates an example of an end effector in accordance with the present invention;

FIG. 5 further illustrates an example of an end effector in accordance with the present invention;

FIG. 6 further illustrates an example of part of an end effector in accordance with the present invention;

FIG. 7 illustrates a further example of an end effector in accordance with the present invention.

### DETAILED DESCRIPTION OF THE EMBODIMENTS

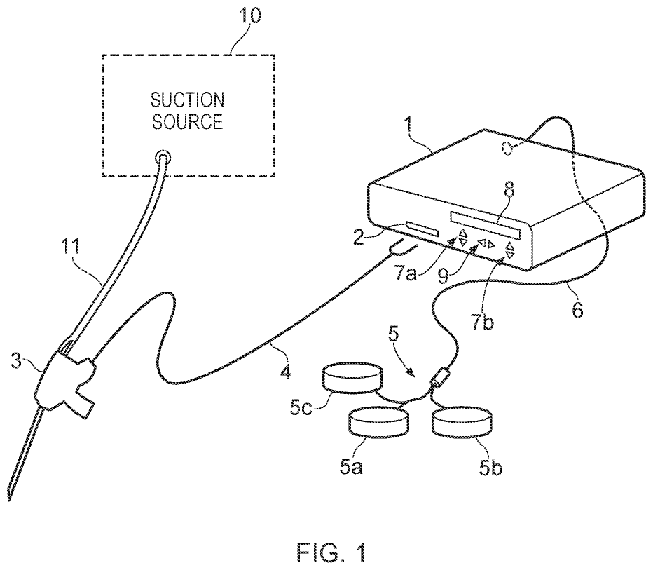

FIG. 1 shows an electrosurgical apparatus including an electrosurgical generator 1 having an output socket 2 that provides a radio frequency (RF) output (e.g. a RF power signal), via a connection cord 4, to an electrosurgical instrument 3 having an end effector that may be configured to provide a mechanical shaving function, as well as an electrosurgical cutting and coagulation functions. The instrument 3 has a suction tube 14 which is connected to a suction source 10. Activation of the generator 1 may be performed from the instrument 3 via a handswitch 12a on the handpiece 12 of the instrument 3, or by means of a footswitch unit 5 connected separately to the rear of the generator 1 by a footswitch connection cord 6. In the illustrated embodiment, the footswitch unit 5 has three footswitches 5a, 5b and 5c for selecting a mechanical shaving mode, a coagulation mode, or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between different modes.

As described above, the electrosurgical instrument 3 may be a dual sided (or an opposite sided) RF shaver device. In this respect, the main RF componentry and the manual shaving/cutting componentry of the instrument 3 can be provided on opposite sides of a distal end portion of the instrument 3. The structure of the distal end of the instrument is described in more detail below.

In known RF electrosurgical instruments that implement a return electrode in the form of an outer shaft, the insulating sleeve used to define the RF return area typically leaves a blunt edge that can catch on other surfaces during use. This can result in damage to the entry site on the patient, and if the material erodes a sufficient amount, can lead to material being left in the patient after the instrument is removed. The present disclosure therefore seeks to address this problem by providing a lead-in feature on the electrode assembly, to gradually increase the diameter of the outer shaft to that of the insulation diameter. This would prevent a user catching this edge on the cannula, and therefore reduce the force needed to insert the device, and reduce the risk of damaging the insulation or hurting the patient.

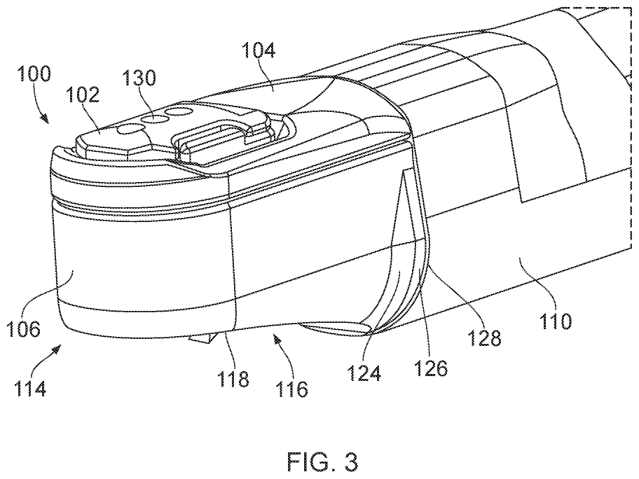

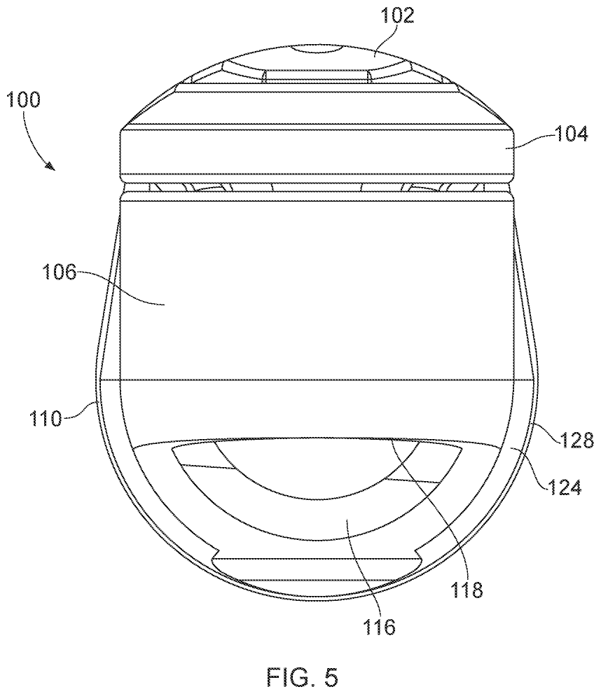

FIGS. 3 to 6 illustrate an example of how the lead-in feature may be implemented in an RF shaving device, in which a mechanical cutting component is located on the opposite side to the active tip. FIG. 3 shows the electrode assembly of an end effector 100 (referred to herein as an RF tip) of an electrosurgical instrument. The end effector 100 comprises an active electrode 102 for tissue treatment, also referred to as the “active tip”, an insulating casing 104 arranged to receive the active electrode 102, and a return electrode component 106. The insulating casing 104 is provided between the active electrode 102 and the return electrode component 106 to physically separate those components, and may be formed from any suitable material, such as ceramic or a polymer. The active electrode 102 and return electrode 106 may be formed from any suitable metal, for example, copper, stainless steel, tungsten or an alloy of tungsten and platinum.

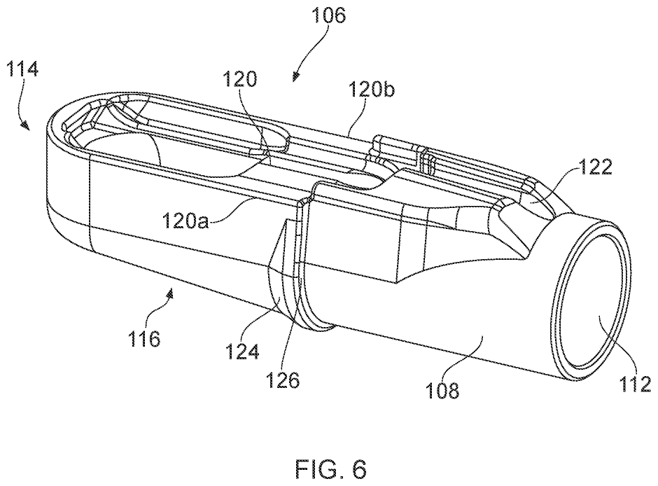

The return electrode component 106 is shown in more detail in FIG. 6. The return electrode component 106 is formed as a moulded component comprising a generally cylindrical shaft 108 around which an insulating sleeve 110 (as seen in FIG. 3) may be provided to secure the return electrode component 106 to the rest of the instrument 3, the shaft 108 providing the return path for the RF power signal. It will be appreciated that the shaft 108 may be electrically and mechanically coupled (for example, by some suitable joining method such as welding) to a separate shaft component (not shown) that extends from the hand piece 12 of the instrument 3, with the insulating sleeve 110 being formed over the shaft 108 of the return electrode component 106 and the separate outer shaft. Alternatively, the shaft 108 may extend all the way down to the hand piece 12, where it is then electrically and mechanically coupled thereto.

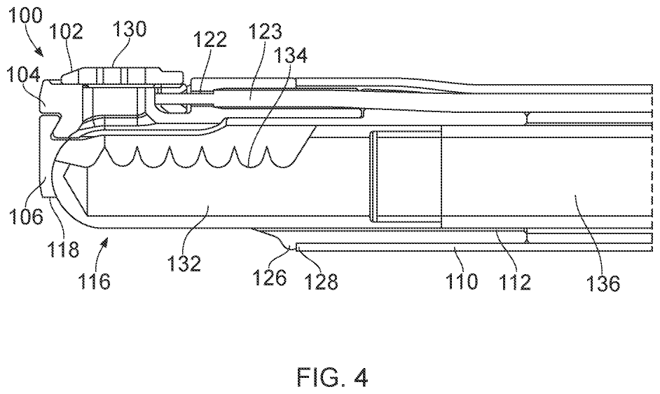

The shaft 108 comprises a lumen 112 that provides a hollow space for receiving an inner blade arrangement 132, as shown in more detail in FIG. 4. The inner blade arrangement 132 comprises a rotating inner blade 134 configured to provide a mechanical cutting mechanism and an inner lumen 136 that provides a suction channel for transporting fluids from tissue treatment site. In this respect, the active electrode 102 may have one or more apertures 130 for providing access to the inner lumen 136. The distal end region 114 of the return electrode 106 comprises an opening 116 that provides a window to the rotating inner blade 134, wherein the opening 116 may also comprise a sharpened edge 118 that acts as a static blade to provide a further cutting function. The return electrode component 106 further comprises a mating surface 120 for receiving the insulating casing 104 and active electrode 102 arrangement, along with any other components used for retaining the active electrode 102 within the insulating casing 104. A channel 122 may also be provided for receiving an electrical conductor 123 that extends along the shaft of the instrument 3 to the hand piece 12 for connection to the generator 1, to thereby deliver the RF power signal to the active electrode 102.

The distal end region 114 of the return electrode component 106 is further provided with lead-in feature in the form of a sloped surface 124 around at least portion of its circumference, for example, approximately 75% of its circumference extending from one side 120a of the mating surface 120 to the opposing side 120b, such that the profile of the distal end region 114 extends beyond that of the shaft 108, and such that it forms an edge 126 facing in the proximal direction (i.e., towards the shaft 108). In this respect, the protruding edge 126 also extends around at least portion of the circumference of the return electrode component, for example, approximately 75% of the circumference. When the insulating sleeve 110 is assembled, as shown in FIGS. 3 to 5, the edge 128 of the sleeve 110 abuts the edge 126 of the distal end region 114 of the return electrode 106 such that the sloped surface 124 and the outside of the insulating sleeve 110 form a smooth outer surface. That is to say, the sloped surface 124 gradually increases the outer diameter of the distal end region 114 of the return electrode 106 such that it matches the outer diameter of the insulating sleeve 110. As such, no overhanging edge or lip is formed by the insulating sleeve 110 that can catch on other surfaces during use.

The return electrode component 106 may be manufactured by any suitable means. One particularly advantageous manufacturing method is Metal Injection Moulding (MIM), which would allow the lead-in feature (e.g., the sloped surface 124) to be easily moulded into the return electrode component 106 without adding significant cost to the return electrode 106 or overall assembly of the end effector 100. Alternatively, the return electrode component 106 may be machined or 3D printed.

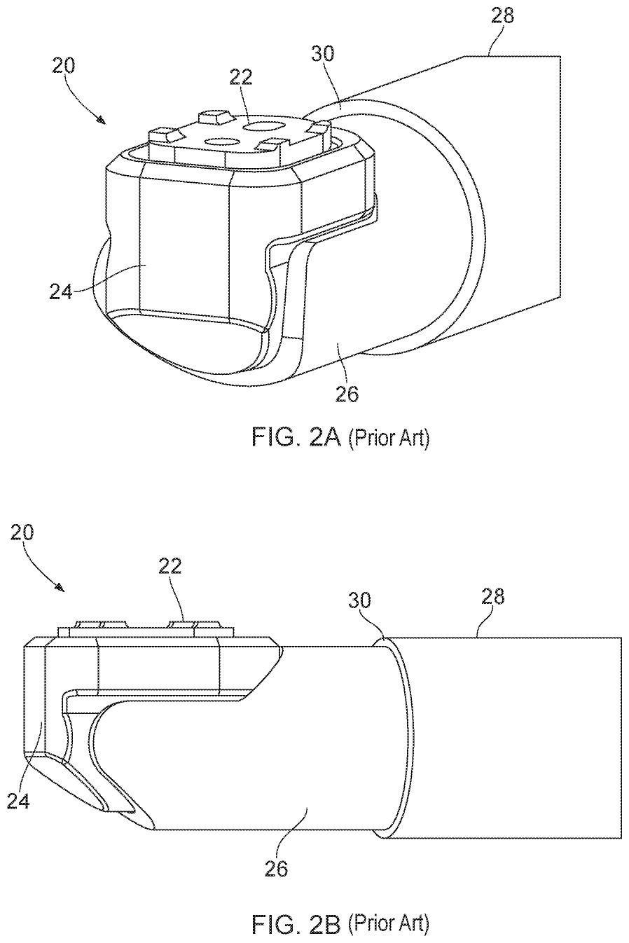

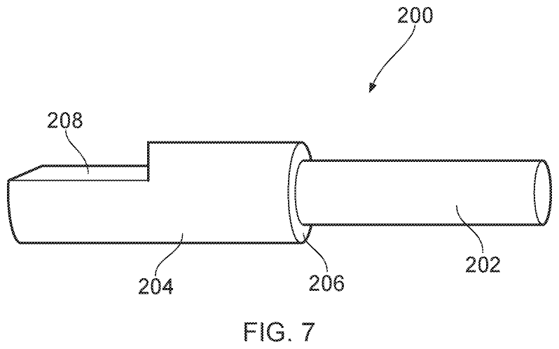

Whilst the example described with reference to FIG. 3-6 shows a single integrally moulded return electrode component 106 that acts as a static blade 118, with a lumen 112 for receiving an inner blade arrangement 132, an RF return electrode shaft 108, and a mating surface 120 for the insulating casing 104 and active electrode 102 arrangement, it may be appreciated that the return electrode may comprise a shaft portion only, such as that shown in FIGS. 2A and 2B. In such cases, as illustrated by FIG. 7, the lead-in feature may be formed by providing a large metal shaft 200 and then machining a portion the shaft 200 down to leave a first portion 202 of smaller diameter and a second portion 204 of larger diameter, with a protruding edge 206 for mating with the end of the insulating sleeve. A further cut-out 208 may be formed for receiving further components of the end effector, such as an active electrode and insulating casing (not shown). Alternatively, in another arrangement, the second portion 204 may be the insulating casing placed between the active electrode (not shown) and the return electrode shaft 202, the insulating casing 204 having a larger diameter than the return electrode shaft 202 to provide the lead-in feature for the insulating sleeve.

As another example, a separate lead-in component may be assembled onto the return shaft or insulating casing, and then welded or bonded in place,

Various further modifications to the above-described embodiments, whether by way of addition, deletion or substitution, will be apparent to the skilled person to provide additional embodiments, any and all of which are intended to be encompassed by the appended claims.

The electrosurgical instrument and/or end effector disclosed herein can be designed to be disposed of after a single use, or they can be designed to be used multiple times. In either case, however, the electrosurgical instrument and/or end effector can be reconditioned for reuse after at least one use. Reconditioning can include a combination of the steps of disassembly of the electrosurgical instrument, followed by cleaning or replacement of particular pieces, and subsequent reassembly. In particular, the electrosurgical instrument can be disassembled, and any number of particular pieces or parts of the device (such as the end effector) can be selectively replaced or removed in any combination. Upon cleaning and/or replacement of particular parts, the electrosurgical instrument can be reassembled for subsequent use either at a reconditioning facility, or by a surgical team immediately prior to a surgical procedure.

Those of ordinary skill in the art will appreciate that the reconditioning of an electrosurgical instrument can utilize a variety of different techniques for disassembly, cleaning/replacement, and reassembly. Use of such techniques, and the resulting reconditioned electrosurgical instrument, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The electrosurgical instrument may also be sterilized using any other technique known in the art, including but limited to beta or gamma radiation, ethylene oxide, or steam.

There follows a set of numbered features describing particular embodiments of the invention. Where a feature refers to another numbered feature then those features may be considered in combination.

1. An end effector for an electrosurgical instrument, comprising:
an electrode assembly for delivering a radio-frequency (RF) power signal to a surgical site, the electrode assembly comprising an active electrode and a return electrode, wherein the electrode assembly comprises at least a first portion having a first outer diameter a second portion having a second outer diameter, the first outer diameter being larger than the second outer diameter; and
an insulating sleeve configured to be received by the second portion of the electrode assembly, the insulating sleeve having an outer diameter substantially the same as the first outer diameter.

2. An end effector according to feature 1, wherein the return electrode comprises the first portion having the first outer diameter and the second portion having the second diameter.

3. An end effector according to feature 2, wherein the return electrode comprises a lead-in feature between the first portion and the second portion to thereby provide a difference in outer diameter.

4. An end effector according to features 2 or 3, wherein the return electrode comprises a protruding edge formed between the first portion and the second portion.

5. An end effector according to feature 4, wherein the protruding edge extends around at least a portion of a circumference of the return electrode.

6. An end effector according to features 4 or 5, wherein the protruding edge extends around up to about 75% of the circumference of the return electrode.

7. An end effector according to any of features 4 to 6, wherein an end of the insulating sleeve is configured to mate with a surface of the protruding edge.

8. An end effector according to any of features 4 to 7, wherein the first portion of the return electrode comprises a sloped surface, the sloped surface forming the protruding edge.

9. An end effector according to feature 1, wherein the electrode assembly further comprises an insulating element arranged between the active electrode and the return electrode, wherein the insulating element comprises the first portion having the first outer diameter and the return electrode comprises the second portion having the second outer diameter.

10. An end effector according to any of features 2 to 8, wherein the electrode assembly further comprises an insulating element arranged between the active electrode and the return electrode.

11. An end effector according to features 9 or 10, wherein the insulating element is formed of a ceramic or a polymer.

12. An end effector according to any preceding feature, further comprising a rotatory shaver arrangement.

13. An end effector according to any preceding feature, wherein the insulating sleeve is heat-shrink wrapped around the second portion of the electrode assembly.

14. An end effector according to any preceding feature, wherein the insulating sleeve comprises a polymer material.

15. An end effector according to feature 14, wherein the polymer material comprising polyvinylidene fluoride.

16. An end effector according to any preceding feature, wherein the return electrode is manufactured by a process of metal injection moulding.

17. An end effector according to any preceding feature, wherein the active electrode comprises an aperture for providing access to a suction channel extending through the insulating element to a lumen configured to carry fluid from the surgical site.

18. An end effector according to feature 17, wherein the lumen is connectable to a suction tube for connecting to a suction source.

19. An electrosurgical instrument, comprising:
a hand-piece;
one or more user-operable buttons on the handpiece for operably controlling the instrument; and
an operative shaft, having RF electrical connections, and drive componentry for an end effector, the electrosurgical instrument further comprising an end effector according to any of the preceding features, the active electrode and the return electrode being connected to the RF electrical connections.

20. An electrosurgical system, comprising:
an RF electrosurgical generator;
a suction source; and
an electrosurgical instrument according to feature 19, the arrangement being such that in use the RF electrosurgical generator supplies an RF coagulation or ablation signal via the RF electrical connections to the active electrode and the return electrode.

1. An end effector for an electrosurgical instrument, comprising:
an electrode assembly for delivering a radio-frequency (RF) power signal to a surgical site, the electrode assembly comprising an active electrode and a return electrode, wherein the return electrode comprises:
a distal region, wherein a portion of the distal region comprises a sloped surface, the sloped surface forming a protruding edge at a first end of the distal region, and the protruding edge having a first outer diameter; and
a proximal region extending from the first end of the distal region, the proximal region having a second outer diameter, the second outer diameter being smaller than the first outer diameter; and
an insulating sleeve configured to be received by the proximal region of the return electrode, the insulating sleeve having an outer diameter substantially the same as the first outer diameter.

2. An end effector according to claim 1, wherein the protruding edge extends around at least a portion of a circumference of the return electrode.

3. An end effector according to claim 1, wherein the protruding edge extends around up to about 75% of the circumference of the return electrode.

4. An end effector according to claim 1, wherein an end of the insulating sleeve is configured to mate with a surface of the protruding edge.

5. An end effector according to claim 1, wherein the electrode assembly further comprises an insulating element arranged between the active electrode and the return electrode.

6. An end effector according to claim 5, wherein the insulating element is formed of a ceramic or a polymer.

7. An end effect according to claim 1, further comprising a rotatory shaver arrangement.

8. An end effector according to claim 1, wherein the insulating sleeve is heat-shrink wrapped around the proximal region of the return electrode.

9. An end effector according to claim 1, wherein the insulating sleeve comprises a polymer material.

10. An end effector according to claim 9, wherein the polymer material comprising polyvinylidene fluoride.

11. An end effector according to claim 1, wherein the active electrode comprises an aperture for providing access to a suction channel extending through the insulating element to a lumen configured to carry fluid from the surgical site.

12. An end effector according to claim 11, wherein the lumen is connectable to a suction tube for connecting to a suction source.

13. An electrosurgical instrument, comprising:
a hand-piece;
one or more user-operable buttons on the handpiece for operably controlling the instrument; and
an operative shaft, having RF electrical connections, and drive componentry for an end effector, the electrosurgical instrument further comprising an end effector according to claim 1, the active electrode and the return electrode being connected to the RF electrical connections.

14. An electrosurgical system, comprising:
an RF electrosurgical generator;
a suction source; and
an electrosurgical instrument according to claim 13, the arrangement being such that in use the RF electrosurgical generator supplies an RF coagulation or ablation signal via the RF electrical connections to the active electrode and the return electrode.
